(19) United States
(12) Patent Application Publication
(10) Pub. No.: US 2025/0114012 A1
(43) Pub. Date: Apr. 10, 2025

(54) MEDICAL SYSTEM AND EMTHOD FOR DETERMINING A POSITION OF A PUNCTURE POINT

(71) Applicant: Hiba Pictet, Collonge Bellerive (CH)

(72) Inventors: Hiba Pictet, Collonge Bellerive (CH); Eric Grenet, Neuchâtel (CH); Clément Kündig, Bussigny (CH); Pedram Pad, Lausanne (CH); Engin Türetken, Ecublens (CH)

(21) Appl. No.: 18/832,278

(22) PCT Filed: Jan. 23, 2023
(86) PCT No.: PCT/IB2023/050548

§ 371 Date: Jul. 23, 2024

(30) Foreign Application Priority Data
Jan. 24, 2022 (CH) 000068/2022

(51) Int. Cl. A61B 5/06; A61B 5/00

(52) U.S. Cl. CPC A61B 5/065; A61B 5/0077; A61B 5/489

(57) ABSTRACT

A medical system includes: a device to be placed at a distance from the puncture zone including: first illumination source arranged to illuminate the puncture zone, and a stereoscopic optical sensor arranged to take first images of the blood vessel, a computing module arranged to: calculate a 3D mapping of the blood vessel based on said first images, calculate a 3D mapping of the puncture zone, determine a 3D position of the piercing point of a needle and/or catheter in the blood vessel, determine a 3D position of the puncture point based on the 3D position of the piercing point and the 3D mapping of the puncture zone, and project the puncture point onto the puncture zone with a projector of the device at its 3D position determined by the computing module. This medical system allows to determine more accurately than known solutions, the position of the puncture point.

### TECHNICAL AREA

The present invention concerns a medical system and a method for determining a position of a puncture point, in particular a puncture point belonging to a puncture zone below which a portion of a blood vessel is located, to facilitate access to this blood vessel by an operator.

### STATE OF THE ART

There are several known solutions for determining the position of a puncture point, i.e. a point at which an operator can insert a needle or catheter into a patient's body, with the aim of accessing a patient's blood vessel.

In some known solutions, a projection of blood vessels can be made on a patient's limb, to assist the operator performing the puncture. An example of such a solution is described in document US2004171923. However, these solutions do not enable a puncture point to be calculated and projected onto the patient's limb.

Other solutions make it possible to calculate and project a puncture point onto a patient's limb, as described for example in EP1981395. These solutions are more interesting, as access to a blood vessel is easier for an operator than with other known solutions.

However, the calculation of the puncture point is not yet sufficiently accurate. As a result, a successful puncture requires several attempts, even with the support of these known solutions. These failures have a certain impact on the patient, especially if the patient is a child.

The document US20120190981 describes a self-contained device for intravenous needle insertion. This device comprises a first unit comprising sensors, such as an NIR source and an NIR camera, a second unit comprising a needle, and a robotic arm. Based on information from the first unit, the arm is moved by actuators to insert the needle into the veins detected by the NIR camera. A perimeter can be displayed on the user's arm, around the area below which the veins are detected. This system does not require any projection of the position of the puncture point in order to show an operator this position, as it is a stand-alone device.

The paper GENERATION AND EVALUATION OF HYPERSPECTRAL 3D SURFACE MODELS BASED ON A STRUCTURED LIGHT SYSTEM WITH HYPERSPECTRAL SNAPSHOT MOSAIC SENSORS, S. HEIST ET AL, Proceedings of SPIE, IEEE, US, Vol. 10667, 14 May 2018, describes a system for generating hyper-spectral 3D surface models, which can find application in the medical field too, for example for vein detection or reconstruction of the 3D surface of a hand. This system, based on hyper-spectral “snapshot” cameras, also includes a “high-speed broadband pattern projector”, in particular of several periodic sinusoidal patterns that vary, using a “GOBO” (“GOes Before Optics”) wheel. This system is not concerned with calculating the position of a puncture.

There is therefore a need for a medical system to determine the position of a puncture point free from the limitations of known systems.

There is also a need for a medical system to determine the position of a puncture point more accurately than known solutions.

### BRIEF SUMMARY OF THE INVENTION

One aim of the present invention is to provide a medical system for determining the position of a puncture point free from the limitations of known systems.

Another aim of the invention is to provide a medical system for determining the position of a puncture point more accurately than known solutions.

According to the invention, these aims are achieved in particular by means of the medical system for determining a position of a puncture according to claim 1, as well as by means of the method according to claim 20.

The medical system according to the invention makes it possible to determine a position of at least one puncture point on a puncture zone below which there is a portion of a blood vessel. The medical system according to the invention therefore enables an operator to correctly introduce a needle or catheter into a patient's blood vessel.

In this context, the expression “puncture zone” refers to a portion of an (external) surface of a patient's skin, for example the skin of a patient's limb. The patient can be a human being, for example and without limitation, a child. The patient can also be an animal.

The system according to the invention comprises a device arranged to be placed at a certain distance from the puncture zone. The fact that the device is placed at a certain distance from the puncture zone, and not in contact with the puncture zone, makes it possible to create a space (between the device and the puncture zone) for the operator and also to avoid contaminating the puncture zone, keeping it sterile after disinfection.

The device according to the invention comprises:
a first illumination source, arranged to illuminate the puncture zone,
a stereoscopic optical sensor arranged to take first images (of at least a portion) of the blood vessel.

Two (or more) (non-stereoscopic) cameras are a non-limiting example of a stereoscopic optical sensor according to the invention.

The system according to the invention also includes a computing module. In one variant, this computing module is separate from the device and connected to the device, for example with communications means (wireless and/or wired). In another variant, this computing module is (at least partially) included in the device. In one variant, this computing module is not monobloc, but comprises several sub-blocks or sub-devices, linked together by communications means (wireless and/or wired).

According to the invention, the computing module is arranged to:
calculate a 3D (i.e. three-dimensional) mapping of the blood vessel, based on the first images;
calculate a 3D mapping of the puncture zone;
determine a 3D position of a needle and/or catheter piercing point in the blood vessel, based on the 3D mapping of the blood vessel;
determine a 3D position of the puncture point, based on the 3D position of the piercing point in the blood vessel and the 3D mapping of the puncture zone.

In one variant, the 3D mapping of the blood vessel, the 3D mapping of the puncture zone, the 3D position of the piercing point in the blood vessel and/or the 3D position of the puncture point can be absolute or relative to each other and/or, for example, to the puncture zone, a needle or a catheter.

In one variant, the optical sensor, the computing module and the projector share the same coordinate system.

In the context of this invention, the expression “3D mapping of the puncture zone” refers to a three-dimensional profiling of the puncture zone. In other words, this 3D mapping makes it possible to determine the 3D position of several points in the puncture zone (or sub-zones of the puncture zone), for example in the reference frame of the stereoscopic optical sensor.

In the context of this invention, the expression “3D blood vessel mapping” refers to a three-dimensional profiling of the subcutaneous blood vessel, e.g. a venous blood vessel. In other words, this 3D mapping makes it possible to determine the 3D position of the blood vessel beneath the puncture zone, for example in the reference frame of the stereoscopic optical sensor.

In the context of this invention, the expression “piercing point” refers to a point where a needle or catheter enters a blood vessel.

According to the invention, the device comprises a projector for projecting the puncture point onto the puncture zone at the 3D position of the puncture point determined by the computing module.

In particular, this solution has the advantage over the prior art of calculating the 3D puncture position more accurately than known solutions. In fact, according to the invention, the 3D position of the puncture point is calculated by also considering the 3D mapping of the puncture zone. The 3D position of the puncture point calculated and displayed in this way is more accurate. As a result, the number of operator failures using the system according to the invention is reduced compared to known solutions. In one variant, the 3D puncture position calculated by the system according to the invention is about 1 mm more accurate in at least one direction, compared with the same position calculated without considering the 3D mapping of the puncture zone.

The 3D mapping of the puncture zone can be calculated by the computing module based on various inputs or information.

In one variant, the device comprises a second illumination source, arranged to project a pattern onto the puncture zone, and the stereoscopic optical sensor is arranged to take second images of the pattern. In this variant, the computing module is arranged to calculate a 3D mapping of the puncture zone based on these second images.

In one variant, the pattern is formed by dots and the computing module is arranged to:
calculate the 3D position of the pattern points based on the second images, and
obtain the 3D mapping of the puncture zone based on the 3D position of the pattern points.

In one variant, the 3D position of the pattern points can be absolute or relative, for example in relation to the puncture zone, to a needle or to a catheter.

In one variant, the dots forming the pattern comprise (at least) one dot (e.g. a central dot), hereinafter referred to as the reference dot, which for example has a different brightness and/or size and/or shape and/or color from the other dots. The reference point could also have a specific known position in the pattern. The reference point could also be a missing point in a pattern. This makes it possible to use this point as a reference, to know which projected point corresponds to which “original” point in the pattern, i.e. which point projected onto a flat surface placed at the same distance from the device's puncture zone.

Several possibilities can be envisaged for projecting a pattern onto the puncture zone. In one variant, the system comprises an optical element in series with the second illumination source and arranged to create the pattern. This optical element can be, for example, an optical diffraction element (e.g. a dot grating, a grid, etc.) or, for example, a structured light projection element.

In another variant, the system comprises several second illumination sources, each second illumination source projecting one or more dots onto the puncture zone, the set of dots forming the pattern.

In other variants, the computing module is arranged to calculate a 3D mapping of the puncture zone based on different inputs from the second images of the pattern taken by the stereoscopic optical sensor.

For example, in another variant, the stereoscopic optical sensor is a first optical sensor, the device and/or system comprises a second optical sensor (not necessarily stereoscopic) arranged to take third images of the puncture zone, and the computing module is arranged to calculate a 3D mapping of the puncture zone based on these third images.

In one variant, the second optical sensor is or comprises at least one RGB sensor, preferably two RGB sensors.

In one variant, the second optical sensor is or comprises a module as an Intel® RealSense™ Depth Module (e.g. D401 and/or D405).

In one variant, the second optical sensor is or comprises a time-of-flight camera.

In one variant, the computing module is arranged to calculate a 3D mapping of the puncture zone based on the output of other modules or sensors that the system and/or the device can comprise, for example a lidar, a radar, a module implementing an artificial intelligence, etc. and/or or any other module or sensor that can send to the computing module the information required to calculate a 3D mapping of the puncture zone. Non-limiting examples of this module and/or sensor include at least one of:
a photometric module,
a module arranged to determine a 3D mapping of the puncture zone from a shading and/or from a polarization,
a photometric stereo module,
a module arranged to determine a 3D mapping of the puncture zone with geometric reconstruction techniques,
a multi-view stereo module,
an active stereo module,
a module arranged to determine a 3D mapping of the puncture zone with structured light,
a module arranged to determine a 3D mapping of the puncture zone with modulated light,
a module arranged to determine a 3D mapping of the puncture zone with conoscopic holography,
a module arranged to determine a 3D mapping of the puncture zone from shadows, contours, texture, pattern, movement, silhouette and/or focus
a module based on stereopsis,
a stereoscopic sensor and/or an inverted depth sensor,
a time-of-flight camera,
a module arranged to determine a 3D mapping of the puncture zone with a lidar system,
a module arranged to determine a 3D mapping of the puncture zone with a radar system,
a module arranged to determine a 3D mapping of the puncture zone with a laser scanner,
a module arranged to determine a 3D mapping of the puncture zone with an ultrasound system,
a module arranged to determine a 3D mapping of the puncture zone with a technique (e.g. of reconstruction and of mapping) based on machine learning,
a module arranged to determine a 3D mapping of the puncture zone with a technique based on simultaneous localization and mapping.

In this context, the expression “module implementing an artificial intelligence” refers to a module arranged to simulate human thinking capability and/or human behavior.

In this context, the expression “module arranged to determine a 3D mapping of the puncture zone with a technique based on machine learning” designates a module that must be trained to learn, i.e. to progressively improve performance on a specific task, in particular the determination of a 3D mapping of the puncture zone.

In another variant, the computing module is arranged to determine the distance between the device and the puncture zone, for example based on the second images, in particular based on the reference point.

In one variant, the system can comprise alarm means, for example visual, audible and/or vibratory means for signalling to the operator whether or not this distance belongs to a range of distances enabling the system to operate. In another variant, these alarm means comprise the same projector, which in this case is arranged to project onto the puncture zone also information to signal to the operator whether or not this distance belongs to the range of distances enabling the system to operate. Using the projector as an alarm means enables the operator to receive information, while keeping his attention on the puncture zone.

In one variant, the computing module is arranged to determine in real time a 3D position of a needle and/or of a catheter based on the first images. This 3D position can be absolute or relative, for example in relation to the puncture zone.

In one variant, the computing module is arranged to determine in real time the orientation of a needle and/or of a catheter, based on the first images. This orientation can be absolute or relative, for example in relation to the puncture zone.

In one variant, the computing module is arranged to determine the position of the piercing and/or puncture point also based on the 3D position and/or orientation of the needle and/or of the catheter.

In one variant, the computing module is arranged to determine and/or to modify in real time the position of the puncture point, so that the puncture point and the piercing point belong to a straight line corresponding to the main direction of the needle or of the catheter.

In one variant, if the main direction of the needle or of the catheter changes (e.g. because of a movement by the operator), the computing module redetermines the 3D position of the puncture point so that the puncture point and the piercing point belong to a straight line corresponding to the new main direction of the needle or of the catheter. In this case, the projector projects a new puncture point onto the puncture zone, corresponding to the new position determined by the computing module.

Once the needle or the catheter has entered the patient's skin, it is preferable that the direction of the needle or catheter is not changed by the operator. Alarm means can be used to signal the operator to maintain this direction.

In one variant, the computing module is arranged to determine the angle between the needle or the catheter and the puncture zone and check whether this angle belongs to a determined range. In one variant, the alarm means are arranged to signal to the operator whether or not this angle belongs to this range and, if necessary, give instructions to the user on how to change this angle, so that it can belong to the desired range.

In the variant that uses the second images of the pattern to calculate the 3D mapping of the puncture zone, the stereoscopic optical sensor is arranged to alternately take a (first) image to determine the 3D mapping of the blood vessel and/or of the needle and/or of the catheter, and a (second) image to obtain the 3D mapping of the puncture zone.

In one variant, the alarm means are arranged to confirm to the operator the entry of the needle and/or of the catheter into the blood vessel. In one variant, the alarm means are arranged to warn the operator if he is damaging the blood vessel, for example by crossing it from one side to the other.

In one variant, the computing module is arranged to differentiate the shadow of the needle and/or of the catheter on the puncture zone from a blood vessel. In one variant, the computing module is arranged to differentiate hairs and/or tattoos and/or other visual artifacts on the piercing area, from a blood vessel.

In one variant, when the needle and/or the catheter touches the patient's skin, the computing module is arranged to stop calculating the 3D mapping of the puncture zone. This is because the 3D mapping is modified by the contact of the needle and/or catheter with the puncture zone.

In one variant, the computing module is arranged to follow the course of the needle and/or of the catheter through the patient's body to entry into the blood vessel based on the first images, the alarm means being arranged to indicate to the operator when to stop and/or warn him upstream if necessary.

In one variant, the first illumination source and/or the second illumination source is/are arranged to emit a spectrum in the NIR and/or IR band, optionally to emit several different wavelengths in the NIR and/or IR band. The combination of different wavelengths increases the quality of blood vessel detection. In one variant, the first illumination source is arranged to emit the following two wavelengths: 850 nm and 940 nm.

In one variant, the first illumination source and/or the second illumination source is/are arranged to emit a spectrum in the visible or UV band, optionally to emit several different wavelengths in the visible and/or UV band, in order to enable the computing module to improve the 3D mapping of the blood vessel and/or to filter out visual artifacts such as hairs, pimples or tattoos.

In one variant, the computing module is arranged to combine (first) images of different wavelengths to increase the quality of detection of the blood vessel and/or its 3D mapping.

In one variant, the computing module is arranged to (re)-determine the 3D position of the piercing point also based on the 3D mapping of the puncture zone. This can be useful when the puncture point position determined based on a (first) piercing point position is not optimal, for example because it is at a scar, a wound, a pimple, etc. In this case, the 3D position of the piercing point can be redetermined by the computing module, also considering the 3D mapping of the puncture zone.

In one variant, the needle and/or the catheter comprises means enabling the computing module to track its movement outside and/or inside the puncture zone, for example a light source arranged to illuminate its tip.

In one variant, the projector for projecting onto the puncture zone the puncture point or a second projector of the device are arranged to project onto the puncture zone or another area of the patient an image or video, to distract the patient during the puncture.

The present invention also relates to a method for determining a position of a puncture point on a puncture zone beneath which a blood vessel is located, comprising the steps of
placing a device at a certain distance from the puncture zone,
illuminating the puncture zone with a first illumination source of the device,
taking first images of the blood vessel with a stereoscopic optical sensor of the device,
calculating a 3D mapping of the blood vessel based on the first images, with a computing module connected to the device and/or included in the device,
calculating a 3D mapping of the puncture zone with the computing module,
determining with the computing module a 3D position of a needle and/or of a catheter piercing point in the blood vessel,
determining with the computing module a 3D position of the puncture point, based on the 3D position of the piercing point in the blood vessel and the 3D mapping of the puncture zone,
projecting with a projector of the device the puncture point onto the puncture zone at its 3D position determined by the computing module.

In one variant, the method comprises the steps of:
projecting a pattern onto the puncture zone with a second illumination source of the device,
taking second images of the pattern with the stereoscopic optical sensor of the device,
calculating with the computing module a 3D mapping of the puncture zone based on the second images.

In another variant, the method comprises the steps of:
taking third images of the puncture zone with a second optical sensor of the device,
calculating with the computing module a 3D mapping of the puncture zone based on the third images.

### BRIEF DESCRIPTION OF FIGURES

Examples of implementation of the invention are shown in the description illustrated by the attached figures in which:

FIG. 1 illustrates a cross-sectional view of one embodiment of a medical system, according to one embodiment of the invention.

FIG. 2 illustrates a top view of one embodiment of the pattern projected onto a flat surface by the second illumination source of the medical system, in one embodiment of the invention.

FIG. 3 illustrates a top view of a portion of one embodiment of the pattern shown in FIG. 2, as projected onto a puncture zone by the second illumination source of the medical system, in one embodiment of the invention.

FIG. 4 schematically illustrates a cross-sectional view of a puncture zone with a syringe.

### EXAMPLE(S) OF EMBODIMENT OF THE INVENTION

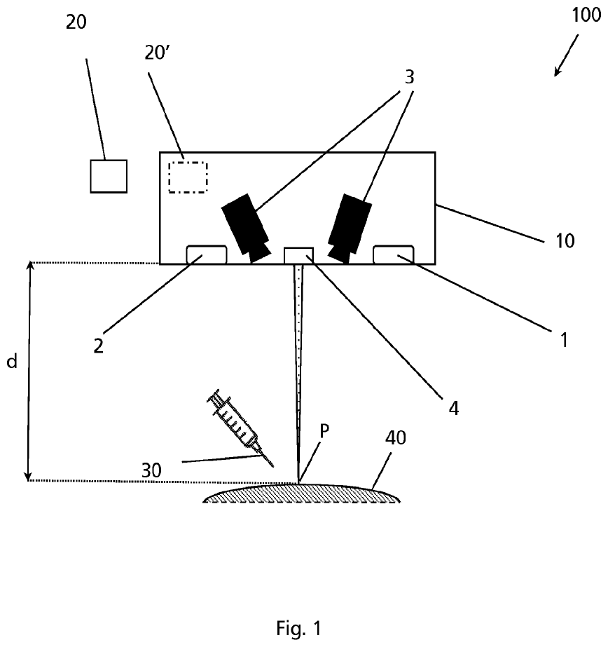

FIG. 1 illustrates a cross-sectional view of one embodiment of a medical system 100, according to one embodiment of the invention.

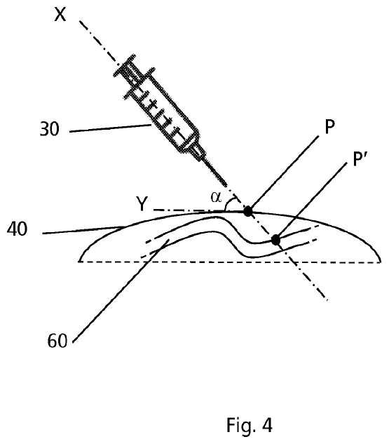

The medical system 100 can determine the position of (at least) one puncture point P on a puncture zone 40, below which there is a portion of a blood vessel 60, visible for example in FIG. 4. The medical system 100 according to the invention can calculate the position of several (optimal) puncture points P on a puncture zone 40, enabling the operator to choose one of them. The dimensions and proportions of the elements illustrated in FIGS. 1 and 4 do not necessarily correspond to the actual ones.

In this context, the expression “puncture zone” refers to a portion of an (external) surface of a patient's skin, for example the skin of a patient's limb, such as the arm. The dimensions of this “puncture zone” lie, for example and without limitation, in the range 1 cm² to 100 cm².

As can be seen from FIG. 1, this puncture zone 40 is not necessarily flat. In fact, it usually has (at least) one curvature. In known solutions, the fact that the puncture zone is not necessarily flat or plane is not taken into account, which explains the lack of precision in the calculated puncture point position. Once the 3D position of the piercing point has been determined, the invention also considers the 3D mapping of the puncture zone when calculating the 3D position of the puncture point, which is therefore more accurate than with known solutions.

As shown in FIG. 1, the system 100 according to the invention comprises a device 10 arranged to be positioned at a certain distance d from the puncture zone 40. In one variant, this distance d is in the range 20 mm to 200 mm. For example, and without limitation, the device 10 comprises an arm enabling it to be positioned at distance d from the puncture zone 40. The fact that the device 10 is positioned at a certain distance d from the puncture zone 40, and not in (at least partial) contact with the puncture zone 40, makes it possible to create a space for the operator between the device 10 and the puncture zone 40, and also to avoid contaminating the puncture zone 40, by keeping it sterile after disinfection.

As shown in FIG. 1, device 10 comprises (at least) a first illumination source 1, arranged to illuminate the puncture zone. In one variant, the first illumination source 1 is arranged to emit a spectrum in the NIR (Near Infra-Red) and/or IR (Infra-Red) band, possibly to emit several different wavelengths in the NIR and/or IR band. In one variant, the first illumination source 1 is arranged to emit the following two wavelengths: 850 nm and 940 nm. The combination of these two wavelengths enhances the quality of blood vessel detection. In another variant, other wavelengths, in the visible and/or in the UV (UltraViolet), will be used to eliminate certain artifacts, such as hairs or tattoos (not illustrated) on the puncture zone 40, and/or enrich/improve the blood vessel signal and the quality/precision of its 3D mapping. In one (non-limiting) variant, the first illumination source 1 is a laser.

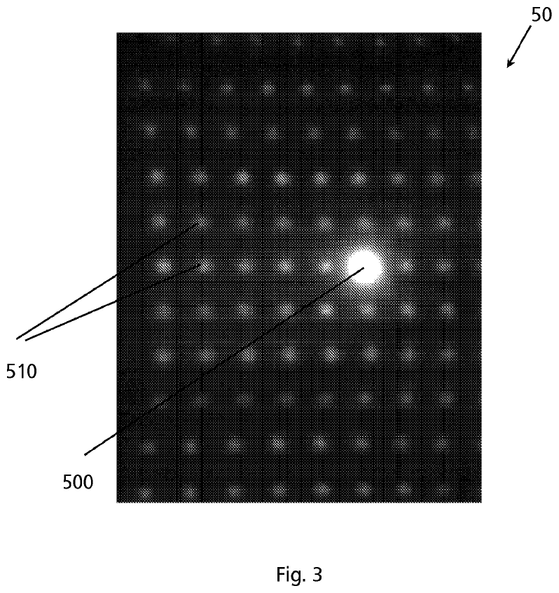

In the variant shown in FIG. 1, the device 10 comprises (at least) a second illumination source 2, arranged to project a pattern 50 (visible, for example, on FIG. 3) onto the puncture zone 40. In a (non-limiting) variant, the second illumination source 2 is also a laser. In one variant, the second illumination source 2 is arranged to emit a spectrum in a band invisible to the human eye, for example in the NIR and/or IR band.

In the variant shown in FIG. 1, the device 10 comprises a stereoscopic optical sensor 3 arranged to take first images (of at least a portion) of the blood vessel and to take second images (of at least a portion) of the pattern. The two (non-stereoscopic) cameras shown in FIG. 1 are a non-limiting example of a stereoscopic optical sensor 3 according to the invention.

The system 100 according to the invention also includes a computing module 20, 20′. In one variant, this computing module 20 is separate from the device 10 and connected to the device, for example via communications means (wireless and/or wired). In another variant, this computing module 20′ is (at least partially) included in the device 10. In one variant, this computing module 20, 20′ is not monobloc, but comprises several sub-blocks or sub-devices, interconnected by communications means (wireless and/or wired).

In one variant, the optical sensor 3, the computing module 20, 20′ and the projector 4 share the same coordinate system.

In the variant shown in FIG. 1, the computing module 20, 20′ is arranged to:
calculate a 3D mapping of the blood vessel, based on the first images,
calculate a 3D mapping of the puncture zone 40, based on the second images,
determine the 3D position of the optimal piercing point P′ of the needle and/or of the catheter in the blood vessel,
determine a 3D position of the puncture point P, based on the 3D position of the piercing point P′ of the needle and/or of the catheter in the blood vessel and the 3D mapping of the puncture zone,
project the puncture point P onto the puncture zone with a projector of the device, at its previously determined 3D position.

According to the invention, the 3D mapping of the puncture zone can be calculated by the computing module 20, 20′ based on other inputs or information, different from the pattern second images.

In one variant, the device comprises a second illumination source, arranged to project a pattern onto the puncture zone, and the stereoscopic optical sensor is arranged to take second images of the pattern. In this variant, the computing module is arranged to calculate a 3D mapping of the puncture zone based on these second images.

In other variants, the computing module 20, 20′ is arranged to calculate a 3D mapping of the puncture zone 40 based on different inputs from the second images of the pattern taken by the stereoscopic optical sensor 3.

For example, in another variant, the stereoscopic optical sensor 3 is a first optical sensor, the device 10 and/or system 100 comprises a second optical sensor (not shown) arranged to take third images of the puncture zone 40. In this variant, the computing module 20, 20′ is arranged to calculate a 3D mapping of the puncture zone 40 based on these third images.

In one variant, the second optical sensor is or comprises at least one RGB sensor, preferably two RGB sensors.

In one variant, the second optical sensor is or comprises a module as an Intel® RealSense™ Depth Module (e.g. D401 and/or D405).

In one variant, the second optical sensor is or comprises a time-of-flight camera.

In one variant, the computing module 20, 20′ is arranged to calculate a 3D mapping of the puncture zone 40 based on the output of other modules or sensors that the system 100 and/or device 10 can comprise, for example a lidar, a radar, a module implementing an artificial intelligence, etc. and/or any other module or sensor that can send to the computing module 20, 20′ the information required to calculate a 3D mapping of the puncture zone 40. In one variant, the computing module 20, 20′ is also arranged to:
supervise the course of the needle and/or of the catheter until the needle tip arrives at the piercing point P′ in the blood vessel, and/or
prevent and/or confirm with alarm means the entry of the needle and/or of the catheter into the blood vessel.

The 3D puncture zone mapping is used to determine the 3D position of several points in the puncture zone 40 or sub-zones of the puncture zone 40, for example in the reference frame of the stereoscopic optical sensor 3. In one variant, these points are those that form the pattern.

In one variant, the 3D mapping of the puncture surface also provides information also on at least one curvature of the puncture zone 40.

According to the invention, the device comprises a projector 4, for example a pico-projector, to project the puncture point P onto the puncture zone 40 at its 3D position determined by the computing module.

According to the invention, the 3D position of the puncture point P is calculated by also considering the 3D mapping of the puncture zone 40. The 3D position of the puncture point P calculated in this way is more accurate than known solutions.

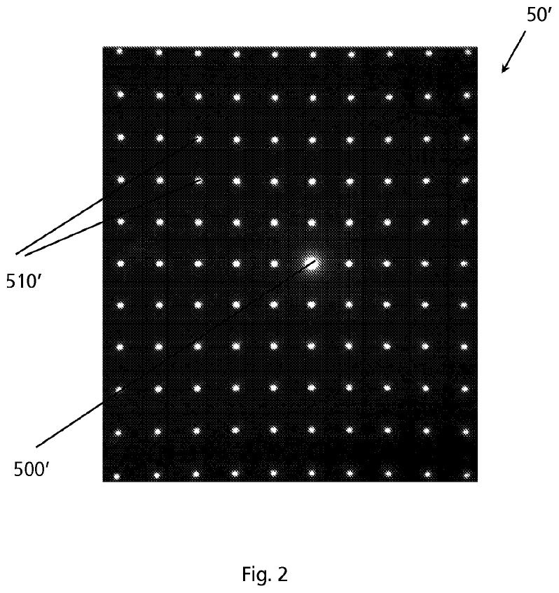

FIG. 2 illustrates a top view of one embodiment of the pattern 50′ as projected onto a flat or planar surface by the second illumination source 2 of the medical system 100, in one variant of the invention. Although in the example shown in FIG. 2 the pattern 50′ is in the form of a matrix or array of dots 500′, 510′, this pattern 50′ is not limitative.

In the variant shown in FIG. 2, the pattern 50′ is formed by several points, of which (at least) one point (e.g. a central point), hereinafter referred to as reference point 500′, has a brightness and/or size and/or shape and/or color different from the other points 510′. The reference point 500′ could also have a specific known position in the pattern 50′ or be a missing point.

FIG. 3 illustrates a top view of the embodiment of a portion of the pattern 50 of FIG. 2, as projected onto a puncture zone 40 by the second illumination source 2 of the medical system 100 in one variant of the invention. If the puncture zone 40 is not a flat surface, the pattern 50 in FIG. 3 is distorted in relation to the pattern 50′ in FIG. 2. Some points have a different size, position and/or brightness to those in FIG. 2. The reference point 500 indicates which projected point 510 of FIG. 3 corresponds to the “original” point 510′ of FIG. 2.

In one variant, the computing module 20, 20′ is arranged to analyze the image shown in FIG. 3, and thus calculate the 3D position of the points 500, 510 of the pattern 50. In this case, it is possible to obtain the 3D mapping of the puncture zone based on the 3D position of points 500, 510 of pattern 50.

In one variant, the 3D mapping is obtained by the 3D position of the points 500, 510 of the pattern 50, as well as by interpolation between these points 500, 510, thus providing a complete estimate of the 3D profiling of the puncture zone 40, which is accurate for the purposes of the system 100 according to the invention.

Several possibilities can be envisaged for projecting a pattern 50 onto the puncture zone 40. In one variant, the system 100 comprises a (not illustrated) optical element in series with the second illumination source 2 and which is arranged to create the pattern 50. This optical element can be, for example, an optical diffraction element (e.g. a dot grating, a grid, etc.) or, for example, a structured light projection element.

In another variant, the system 100 comprises several second illumination sources 2, each second illumination source 2 projecting one or more dots onto the puncture zone 40, all these dots together forming the pattern 50.

In another variant, the computing module 20, 20′ is arranged to determine the distance d between the device 10 and the puncture zone 40, for example based on the second images, for example based on the reference point 500. In this case, the system 100 can comprise alarm means, for example visual, audible and/or vibratory means to signal to the operator whether or not this distance d belongs to a range of distances enabling the system 100 to operate.

In another variant, these alarm means comprise the same projector 4, which in this case is arranged to project onto the puncture zone 40 also visual information to signal to the operator whether or not this distance d belongs to the range of distances allowing the system 100 to operate. Using the projector 4 as an alarm means enables the operator to receive information, while keeping his attention on the puncture zone 40.

In one variant, the computing module 20, 20′ is arranged to determine in real time the 3D position and/or an orientation of a needle 30 and/or a catheter based on the first images. This 3D position and/or orientation can be absolute or relative, for example in relation to the puncture zone 40.

In one variant, the computing module 20, 20′ is arranged to determine the position of the puncture point P and/or piercing point P′ also based on the 3D position and/or orientation of the needle 30 and/or of the catheter.

In one variant, the alarm means are arranged to project onto the puncture zone 40 also information to signal to the operator whether or not the orientation of the needle 30 and/or of the catheter needs to be modified before puncturing the patient. In one variant, these alarm means are arranged to project onto the puncture zone 40 any information relevant to the correct course of operations, for example the puncture direction (and not just the puncture point), the depth of the piercing point P′, etc.

In one variant, the computing module is arranged to determine and/or modify in real time the position of the puncture point P, so that the puncture point P and the piercing point P′ belong to a straight line corresponding to the main direction X of the needle or of the catheter, as visible for example in FIG. 4.

In one variant, if the main direction X of the needle or of the catheter 30 changes (e.g. because of a movement by the operator), the computing module 20, 20′ redetermines the 3D position of the puncture point P so that the puncture point P and the piercing point P′ belong to a straight line corresponding to the new main direction of the needle or catheter 30. In this case, the projector 4 projects a new puncture point onto puncture zone 40, corresponding to the new position determined by computing module 20, 20′.

Once the needle or the catheter 30 has entered the patient's skin, it is preferable that the direction of the needle or catheter is not changed by the operator. Alarm means can be used to signal the operator to maintain this direction.

In one variant, the computing module 20, 20′ is arranged to determine the angle α (visible in FIG. 4) between the needle or the catheter and the puncture zone 40, or its tangent Y (for example at the puncture point P) if the puncture zone 40 has a curvature.

In this variant, the computing module 20, 20′ is arranged to check whether this angle α belongs to a given range, for example the 20°-40° range. In one variant, the alarm means are arranged to signal to the operator whether or not this angle α belongs to this range and, if necessary, give instructions to the user (e.g. via the alarm means) on how to change this angle α, so that it can belong to the desired range.

In one variant, the stereoscopic optical sensor 3 is arranged to alternately take a (first) image to determine the 3D mapping of the blood vessel and/or of the needle 30 and/or of the catheter, and a (second) image to obtain the 3D mapping of the puncture zone. In this way, the system 100 according to the invention uses a single stereoscopic optical sensor 3, with two quite different illumination sources 1, 2.

In one variant, the alarm means are arranged to warn and/or confirm to the operator the entry of the needle 30 and/or of the catheter into the blood vessel.

In one variant, the computing module is arranged to differentiate the shadow of the needle 30 and/or of the catheter on the puncture zone 40, from a blood vessel.

In one variant, when the needle 30 and/or the catheter touches the puncture zone 40, the computing module 20, 20′ is arranged to stop calculating the 3D mapping of the puncture zone 40. In fact, this 3D mapping of the puncture zone is modified by the contact of the needle 30 and/or of the catheter with the puncture zone 40.

In one variant, the computing module 20, 20′ is arranged to follow the course of the needle 30 and/or of the catheter through the patient's body to entry into the blood vessel based on the first images, the alarm means being arranged to indicate to the operator when to stop and/or warn him upstream if necessary.

In one variant, the computing module determines the optimal piercing point P′ in the blood vessel via algorithms implementing the recommendations and/or experience of professionals (such as nurses, doctors, etc.).

In one variant, the computing module includes an automatic learning module, enabling it, for example, to continuously improve the optimality of the choice of piercing point P′ in the 3D mapping of the blood vessel.

In one variant, the needle and/or the catheter 30 comprises means enabling the computing module 20, 20′ to track its movement outside and/or inside the puncture zone 40, for example a light source arranged to illuminate its tip.

In one variant, the projector 4 for projecting the puncture point P onto the puncture zone 40 or a second projector (not shown) of the device are 10 arranged to project an image or video onto the puncture zone or another area of the patient to distract him during the puncture.

### REFERENCE NUMBERS USED ON FIGURES

1 First source of illumination
2 Second source of illumination
3 Stereoscopic optical sensor
4 Projector
10 Device
20, 20′ Computing module
30 Needle
40 Puncture zone
50,50′ Pattern
60 Blood vessel
100 System
500, 500′ Pattern reference point
510, 510′ Pattern points
d Distance
P Punction point
P′ Piercing point
X Main needle direction
Y Direction of the tangent to the puncture zone at P
α Angle

What is claimed is:

1. A medical system for determining a position of a puncture point on a puncture zone beneath which a blood vessel is located, said system comprising:
a device arranged to be placed at a certain distance from the puncture zone, said device comprising:
a first illumination source, arranged to illuminate the puncture zone,
a stereoscopic optical sensor arranged to take first images of said blood vessel,
a computing module arranged to:
calculate a 3D mapping of said blood vessel based on said first images,
calculate a 3D mapping of the puncture zone,
determine the 3D position of a piercing point of a needle or catheter in the blood vessel,
determine the 3D position of the puncture point, based on the 3D position of the piercing point and the 3D mapping of the puncture zone,
the device comprising a projector for projecting onto the puncture zone said puncture point at the position determined by the computing module.

2. System according to claim 1, the device comprising:
a second illumination source, arranged to project a pattern onto said puncture zone,
the stereoscopic optical sensor being arranged to take second images of the pattern, the computing module being arranged to calculate a 3D mapping of the puncture zone based on said second images.

3. System according to claim 2, wherein the pattern is formed of dots and the computing module is arranged to:
calculate the 3D position of the points of the pattern based on the second images, and
obtain the 3D mapping of the puncture zone based on the 3D position of the points of the pattern.

4. System according to claim 3, in which the dots that form the pattern include a reference dot, for example a dot that has a different brightness and/or size and/or shape and/or color from the other dots.

5. System according to claim 2, comprising an optical element in series with the second illumination source and arranged to create said pattern, and/or wherein the system comprises a plurality of second illumination sources, each second illumination source projecting a dot onto the puncture zone, the set of such dots forming the pattern.

6. System according to claim 1, the stereoscopic optical sensor being a first optical sensor, the device and/or the system comprising a second optical sensor arranged to take third images of the puncture zone, the computing module being arranged to calculate a 3D mapping of the puncture zone based on said third images.

7. System according to claim 1, said computing module being arranged to determine the distance between the device and the puncture zone, the system comprising alarm means for signalling to the operator whether or not this distance belongs to a range of distances allowing the system to operate, these alarm means comprising for example said projector.

8. System according to claim 1, said computing module being arranged to determine in real time a 3D position of a needle and/or of a catheter based on said first images.

9. System according to claim 1, said computing module being arranged to determine in real time an orientation of a needle and/or of a catheter based on said first images.

10. System according to claim 8, said computing module being arranged to determine the position of the puncture point also based on the 3D position and/or orientation of the needle and/or of the catheter.

11. System according to claim 2, wherein the stereoscopic optical sensor is arranged to alternately take a first image to determine the 3D mapping of the blood vessel and/or of the needle and/or of the catheter and a second image to obtain the 3D mapping of the puncture zone.

12. System according to claim 7, the alarm means are arranged to warn and/or confirm to the operator the entry of the needle and/or of the catheter into the blood vessel.

13. System according to claim 1, said computing module being arranged to differentiate the shadow of the needle and/or of the catheter on the puncture zone, from a blood vessel.

14. System according to claim 1, said computing module is arranged to follow the course of the needle and/or of the catheter through the patient's body, till the entry into the blood vessel based on the first images, the alarm means being arranged to indicate to the operator when to stop and/or warn him upstream.

15. System according to claim 1, the first illumination source and/or the second illumination source being arranged to emit a spectrum in the NIR and/or IR band, optionally to emit several different wavelengths in the NIR and/or IR band, for example to emit the wavelengths of 850 nm and 940 nm.

16. System according to claim 1, the first illumination source and/or the second illumination source being arranged to emit a spectrum in the visible or UV band, optionally to emit several different wavelengths in the visible and/or UV band, in order to enable the computing module to improve the 3D mapping of the blood vessel and/or to filter out visual artifacts such as hairs, pimples or tattoos.

17. System according to claim 1, the computing module being arranged to determine and/or modify in real time the position of the puncture point so that the puncture point and the piercing point belong to a straight line corresponding to the main direction of the needle or of the catheter.

18. System according to claim 1, the computing module being arranged to determine the angle between the needle or the catheter and the puncture zone and to check whether this angle belongs to a determined range.

19. System according to claim 1, the projector or a second projector of the device is/are arranged to project onto the puncture zone or onto another area of the patient an image or video in order to distract him during the puncture.

20. Method for determining a position of a puncture point on a puncture zone below which a blood vessel is located, comprising the steps of
positioning a device at a certain distance from the puncture zone,
illuminating the puncture zone with a first illumination source of the device,
taking first images of said blood vessel with a stereoscopic optical sensor of the device,
calculating with a computing module connected to the device and/or included in the device a 3D mapping of said blood vessel based on said first images,
calculating with the computing module a 3D mapping of the puncture zone,
determine with the computing module the 3D position of a piercing point of a needle and/or of a catheter in the blood vessel,
calculate with the computing module a 3D position of the puncture point, based on the 3D position of the piercing point and the 3D mapping of the puncture zone,
projecting with a projector of the device onto the puncture zone said puncture point at its 3D position determined by the computing module.
